(19) United States
(12) Patent Application Publication
(10) Pub. No.: US 2009/0239827 A1
(43) Pub. Date: Sep. 24, 2009

(54) COMPOUNDS HAVING LIPID LOWERING PROPERTIES

(76) Inventors: Esra Ogru, Victoria (AU); Roksan Libinaki, Victoria (AU); Simon Michael West, Victoria (AU)

Correspondence Address: FISH & RICHARDSON PC, P.O. BOX 1022, MINNEAPOLIS, MN, 55440-1022, US

(21) Appl. No.: 11/817,453

(22) PCT Filed: Jan. 6, 2006
(86) PCT No.: PCT/US06/00281

§ 371 Date: Oct. 30, 2008

(30) Foreign Application Priority Data
Mar. 3, 2005 (AU) 2005901013

(51) Int. Cl. A61K 31/665; A61K 31/355; A61P 9/00

(52) U.S. Cl. 514/100; 514/458

(57) ABSTRACT

There is provided a therapy for lowering the blood levels of a lipid selected from the group comprising LDL cholesterol, triglycerides, overall cholesterol and mixtures thereof, the therapy comprising the step of administering an effective amount of one or more phosphate derivatives of one or more electron transfer agents.

### FIELD OF THE INVENTION

The invention relates to a therapy which utilises the ability of modified electron transfer agents to lower the circulating blood levels of one or more of the following lipids: LDL cholesterol, triglycerides and overall cholesterol.

### BACKGROUND OF THE INVENTION

In this specification where a document, act or item of knowledge is referred to or discussed, this reference or discussion is not an admission that the document, act or item of knowledge or any combination thereof was at the priority date, publicly available, known to the public, part of common general knowledge; or known to be relevant to an attempt to solve any problem with which this specification is concerned.

Whilst the following description relates to cardiovascular disease, it is to be understood that this is merely illustrative and that the invention is not limited to cardiovascular disease but that the invention also similarly relates to any condition which involves increased lipid levels.

Cardiovascular disease (CVD) which includes heart disease and stroke is the number one cause of death in Western societies. This is believed to be due to a number of factors including, excessive proliferation of vascular smooth muscle cells (SMC), elevated total cholesterol and low density lipoprotein (LDL) cholesterol. Although changes in lifestyle, including diet and exercise, are recommended first lines of intervention, drug therapy is not only often used, it is also often warranted.

While often thought of as the same thing, heart and cardiovascular disease are different, involving different parts of the body. Heart disease refers only to diseases of the heart and the blood vessel system within the heart. Cardiovascular disease refers to diseases of the heart and diseases of the blood vessel system (arteries, capillaries, veins) within a person's entire body, such as the brain, legs, and lungs. “Cardio” refers to the heart and “vascular” refers to the blood vessel system.

The heart is a strong, muscular pump slightly larger than a fist. It pumps blood continuously through the circulatory system, the network of elastic tubes that allows blood to flow throughout the body. The circulatory system includes two major organs, the heart and lungs, and blood vessels (arteries, capillaries, and veins). Arteries and capillaries carry oxygen- and nutrient-rich blood from the heart and lungs to all parts of the body. Veins carry oxygen- and nutrient-depleted blood back to the heart and lungs. Heart and blood vessel problems do not happen quickly. Over time, the arteries that bring blood to the heart and brain can become blocked from a build up of cells, fat, and cholesterol (plaque). Reduced blood flow to the heart from blockages in the arteries causes heart attacks. Lack of blood flow to the brain from a blood clot, or bleeding in the brain from a broken blood vessel, causes a stroke.

There are many forms of heart and cardiovascular disease, and what follows is a list of the most common of these diseases.
Coronary heart disease (or coronary artery disease).
Angina.
Stroke.
High blood pressure (or hypertension).
Heart failure.

Many things can put a person at risk for heart and cardiovascular disease. The more risk factors (or things that increase risk) a person has, the greater the chance that heart or cardiovascular disease will develop.

Factors include the following:
age;
smoking (active or passive);
high blood pressure;
high blood cholesterol;
physical inactivity;
excessive body weight; and
diabetes.

There are some factors which cannot be controlled such as getting older, family health history, and race. However it is possible to control the three biggest risk factors for heart and cardiovascular disease-smoking, high blood pressure, and high blood cholesterol. Having a low saturated fat, low cholesterol diet and getting regular exercise are excellent health habits. These good health habits will lower blood pressure and keep blood sugar and blood cholesterol levels healthy.

Cholesterol is a fatty substance made by the liver and found in all parts of the body. The body uses cholesterol to produce cell membranes, hormones, vitamin D, and the bile acids that help to digest fat. It takes only a small amount of cholesterol in the blood to meet these needs, and the liver makes all the cholesterol the body needs.

Cholesterol is also accumulated from food. Eating too much cholesterol in animal foods like meats, whole milk dairy products, egg yolks, poultry, and fish can increase cholesterol levels. However, saturated fat in diets is the main culprit that causes cholesterol levels to rise.

Cholesterol travels through the blood in packages called lipoproteins. Low density lipoprotein (LDL) and high density lipoprotein (HDL) are two types of lipoproteins. LDL is often called the “bad” type of cholesterol because it can cause build up and blockage in the arteries that carry blood to the heart. HDL is known as “good” cholesterol because it helps remove cholesterol from the blood, preventing build up and blockage in the arteries.

Medicines used to lower cholesterol levels, if needed, are used along with lifestyle changes. The main goal of cholesterol-lowering treatment is to lower LDL (bad cholesterol) levels enough to reduce the risk of getting heart disease or having a heart attack. There are several types of drugs available for cholesterol lowering, including statins, bile acid sequestrants, nicotinic acid, and fibric acids. Each class of drugs has its own benefits, side effects and cautions.

| | Generic and brand | | Side effects and |
|---|---|---|---|
| Drug class | names | Benefits | cautions* |
| Statins | Atorvastatin (Lipitor) | Reduce LDL and | Upset stomach, gas, |
| | Fluvastatin (Lescol) | triglycerides, and | constipation, abdominal |
| | Lovastatin (Altocor, | moderately increase HDL | pain, cramps, muscle |
| | Mevacor) | | soreness, pain and |
| | Pravastatin (Pravachol) | | weakness, increased |
| | Rosuvastatin (Crestor) | | blood levels of some |
| | Simvastatin (Zocor) | | statins with grapefruit |
| | | | juice consumption |
| Bile acid- | Cholestyramine | Reduce LDL | Constipation, bloating, |
| binding | (Questran) | | nausea, gas |
| resins | Colesevelam (WelChol) | | |
| | Colestipol (Colestid) | | |
| Cholesterol | Ezetimibe (Zetia) | Reduce LDL, slightly | Stomach pain, fatigue |
| absorption | | decrease triglycerides and | |
| inhibitors | | slightly increase HDL | |
| Combination | Ezetimibe/simvastatin | Reduce LDL and | Same as statins and |
| cholesterol | (Vytorin) | triglycerides and | cholesterol absorption |
| absorption | | moderately increase HDL | inhibitors |
| inhibitor and | | | |
| statin | | | |
| Fibrates | Fenofibrate (Lofibra, | Reduce triglycerides and | Gastrointestinal |
| | Tricor) | modestly increase HDL | discomfort, increased risk |
| | Gemfibrozil (Lopid) | | of gallstones |
| Niacin | A variety of prescription | Increase HDL and reduce | Flushing of face and |
| (vitamin B-3, | or over-the-counter | LDL and triglycerides | neck, nausea, vomiting, |
| nicotinic | preparations available in | | diarrhoea, gout, high |
| acid) | three forms: Immediate | | blood sugar, peptic ulcers |
| | release, timed release, | | |
| | extended release | | |

*All types of cholesterol-lowering drugs - with the possible exception of cholesterol absorption inhibitors - may cause liver function abnormalities.

Whilst these cholesterol lowering drugs are very important in reducing the risk of heart disease, there is the downside with all of them that once the desired level has been achieved, it is necessary to continue taking the drugs indefinitely to maintain that level.

There is thus a need for a pharmaceutical substance which can be used to lower lipid levels which has fewer side effects than the current drugs and which does not lead to an indefinite need to administer the drug.

### Tocopherol

Low levels of α-tocopherol (vitamin E) have been associated with increased incidence of coronary heart disease. Conversely, increased intake of α-tocopherol has been shown to have protective effects against heart disease. Since vitamin E is an antioxidant, it is thought to target the cause of atherosclerosis by preventing oxidation of LDL. Studies have also been undertaken to examine potential non-antioxidant mechanisms of vitamin E which could prevent formation of atherosclerotic plaques. Such responses include inhibition of smooth muscle cell proliferation, preservation of endothelial function, inhibition of monocyte-endothelial adhesion, inhibition of monocyte reactive oxygen species and cytokine release, and inhibition of platelet adhesion and aggregation. Clinical trials with vitamin E have however been equivocal in demonstrating treatment of atherosclerosis. Current vitamin E supplements are therefore not a useful clinical option to combat atherosclerosis.

In international patent application no WO 03/026673, there is disclosure that having increased storage levels of vitamins, including tocopheryl phosphate, could be beneficial in alleviating or treating inflammatory conditions such as coronary disease, atherosclerosis and diabetes. However, there is no disclosure of lowering the blood levels of lipids such as cholesterol. The process of inflammation involves a complicated set of pathways. These pathways are not involved in the pathways of lipid-metabolism, cholesterol uptake etc.

Tocopheryl phosphate has also been disclosed in international patent application no WO 2004/064831 as having properties related to inhibiting the proliferation of monocytes/macrophages, proliferation of smooth muscle cells, the expression of CD36 receptors and the uptake of oxidized LDL. However, there is no disclosure of lowering the blood levels of lipids such as cholesterol. There are plenty of studies that have shown that CD36 promotes changes in response to proteins that accumulate in Alzheimer's disease and atherosclerosis. These processes have nothing to do with lipid metabolism. For example, drugs such as Malaria treatments and Alzheimers treatments are aimed at the CD36 expression but do not alter lipid profiles.

### SUMMARY OF THE INVENTION

It has now been surprisingly found that the phosphate derivatives of electron transfer agents are more effective than the non-phosphorylated electron transfer agents at lowering the blood levels of one or more of the following lipids:
LDL cholesterol,
triglycerides, and
overall cholesterol.

According to a first aspect of the invention, there is provided a therapy for lowering the blood levels of a lipid selected from the group comprising LDL cholesterol, triglycerides, overall cholesterol and mixtures thereof, the therapy comprising the step of administering an effective amount of one or more phosphate derivatives of one or more electron transfer agents.

A person skilled in the art will understand that the therapy of the invention will be useful in relation to therapeutic treatment of diseases which are associated with increased blood levels of one or more of the following lipids: LDL cholesterol, triglycerides, and overall cholesterol. Examples of such diseases include, but are not limited to, cardiovascular disease, atherosclerosis, diabetes mellitus, chronic renal disease, primary and secondary hyperlipidemias and dyslipidemia, retinopathies, liver & spleen enlargement and xanthomas.

The invention thus includes a therapy for alleviating symptoms, treating or preventing a disease selected from the group consisting of cardiovascular disease, atherosclerosis, diabetes mellitus, chronic renal disease, primary and secondary hyperlipidemias and dyslipidemia, retinopathies, liver and spleen enlargement, xanthomas and combinations thereof, the therapy comprising administering a pharmaceutical formulation comprising an effective amount of one or more phosphate derivatives of one or more electron transfer agents to a subject having or at risk of developing the disease.

In a further aspect, the invention provides a pharmaceutical composition when used for lowering the blood levels of a lipid selected from the group consisting of LDL cholesterol, triglycerides, overall cholesterol and mixtures thereof, the composition comprising an effective amount of one or more phosphate derivatives of one or more electron transfer agents.

In a further aspect, the invention provides for use of an effective amount of one or more phosphate derivatives of one or more electron transfer agents together with a suitable carrier or diluent in the manufacture of a medicament for lowering the blood levels of a lipid selected from the group consisting of LDL cholesterol, triglycerides, overall cholesterol and mixtures thereof.

In another aspect of the invention, there is provided a therapy for lowering the blood levels of a lipid selected from the group consisting of LDL cholesterol, triglycerides, overall cholesterol and mixtures thereof, the therapy comprising the step of delivering an effective amount of one or more phosphate derivatives of one or more electron transfer agents. In one embodiment of this aspect, the effective amount of one or more phosphate derivatives of one or more electron transfer agents is delivered as a prodrug.

Preferably, the subject is an animal, more preferably the animal is a human.

The term “effective amount” is used herein to refer to an amount which is sufficient to lower the circulating blood levels of one or more of the following lipids: LDL cholesterol, triglycerides, and overall cholesterol. A person skilled in the art will understand that this amount will vary from patient to patient and is usually determined from experience with clinical administration to particular patients.

The term “electron transfer agents” is used herein to refer to the class of chemicals which may be phosphorylated and which (in the non-phosphorylated form) can accept an electron to generate a relatively stable molecular radical or accept two electrons to allow the compound to participate in a reversible redox system. Examples of classes of electron transfer agent compounds that may be phosphorylated include hydroxy chromans including alpha, beta, gamma and delta tocols in enantiomeric and racemic forms; quinols being the reduced forms of vitamin K1 and ubiquinone; hydroxy carotenoids including retinol; calciferol and ascorbic acid. Preferably, the electron transfer agent is selected from the group consisting of tocopherol and other tocols, retinol, vitamin K1 and mixtures thereof.

More preferably, the electron transfer agent is selected from the group consisting of the tocols and mixtures thereof. The tocols include all isomers of derivatives of 6:hydroxy 2:methyl chroman (see structure below) where R₁, R₂ and R₃ may be hydrogen or methyl groups, that is, the α-5:7:8 tri-methyl; β-5:8 di-methyl; γ-7:8 di-methyl; and δ-8 methyl derivatives. In the tocopherols, R₄ is substituted by 4:8:12 tri-methyl tridecyl and includes various stereoisomers and optical isomers (chiral centres are indicted by the *). In the tocotrienols, R₄ is substituted by 4:8:12 tri-methyl trideca-3:7:11 triene and the 2 position may be stereoactive as R or S stereoisomers. Most preferably, the electron transfer agent is selected from the group consisting of α-tocopherol, δ-tocopherol, γ-tocopherol, α-tocotrienol, δ-tocotrienol, γ-tocotrienol and mixtures thereof.

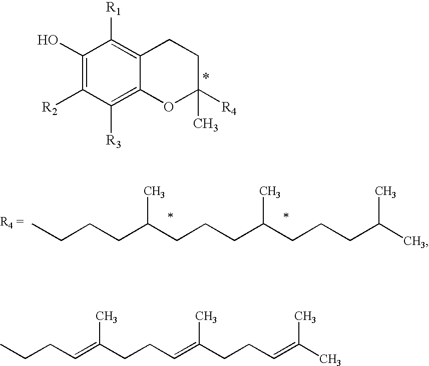

The term “phosphate derivatives” is used herein to refer to compounds covalently bound by means of an oxygen to the phosphorus atom of a phosphate group thus forming a carbon-oxygen-phosphorous bond. The oxygen atom is typically derived from a hydroxyl group on the electron transfer agent. The term includes the acid forms of phosphorylated electron transfer agents, salts of the phosphates including metal salts such as sodium, magnesium, potassium and calcium and any other derivative where the phosphate proton is replaced by other substituents such as ethyl or methyl groups or phosphatidyl groups. The term includes mixtures of phosphate derivatives, especially those which result from phosphorylation reactions, as well as each of the phosphate derivatives alone. For example, the term includes a mixture of mono-tocopheryl phosphate (TP) and di-tocopheryl phosphate (T2P) as well as each of TP and T2P alone. Suitable mixtures are described in international patent application no PCT/AU01/01475.

Preferably, the one or more phosphate derivatives of one or more electron transfer agents is selected from the group consisting of mono-tocopheryl phosphate, di-tocopheryl phosphate, mono-tocotrienyl phosphate, di-tocotrienyl phosphate and mixtures thereof. In one preferred embodiment, the one or more phosphate derivatives of one or more electron transfer agents is a mixture of one or more of mono-tocopheryl phosphate, di-tocopheryl phosphate, mono-tocotrienyl phosphate and di-tocotrienyl phosphate.

In some situations, it may be necessary to use a phosphate derivative such as a phosphatide where additional properties such as increased water solubility are preferred. Phosphatidyl derivatives are amino alkyl derivatives of organic phosphates. These derivatives may be prepared from amines having a structure of R₁R₂N(CH₂)ₙOH wherein n is an integer between 1 and 6 and R₁ and R₂ may be either H or short alkyl chains with 3 or less carbons. R₁ and R₂ may be the same or different. The phosphatidyl derivatives are prepared by displacing the hydroxyl proton of the electron transfer agent with a phosphate entity that is then reacted with an amine, such as ethanolamine or N,N′ dimethylethanolamine, to generate the phosphatidyl derivative of the electron transfer agent. One therapy of preparation of the phosphatidyl derivatives uses a basic solvent such as pyridine or triethylamine with phosphorous oxychloride to prepare the intermediate which is then reacted with the hydroxy group of the amine to produce the corresponding phosphatidyl derivative, such as P cholyl P tocopheryl dihydrogen phosphate.

In some situations, complexes of phosphate derivatives of the electron transfer agents may also be utilized where additional properties such as improved stability or deliverability may be useful. The term “complexes of phosphate derivatives” refers to the reaction product of one or more phosphate derivatives of electron transfer agents with one or more complexing agents selected from the group consisting of amphoteric surfactants, cationic surfactants, amino acids having nitrogen functional groups and proteins rich in these amino acids as disclosed in international patent application no PCT/AU01/01476, incorporated herein by reference. Examples of proteins rich in these amino acids are those proteins having either at least 1 in 62 amino acids as arginine, or at least 1 in 83 histidine, or at least 1 in 65 as lysine, such as the various forms of the protein casein. Other examples include insulin, parathyroid hormone (PTH), glucagon, calcitonin, adrenocorticotropic hormone (ACTH), prolactin, interferon-α and -β and -γ, leutenising hormone (LH) (also known as gonadotropin releasing hormone), follicle stimulating hormone (FSH) and colony stimulating factor (CSF). The amino acid composition of most of these examples is listed in the table.

| Amino acids in protein | Amino acids | Ratio of Total Amino acids |
|---|---|---|
| Insulin | 110 | |
| arg | 5 | 1 in 22 |
| his | 2 | 1 in 55 |
| lys | 2 | 1 in 55 |
| PTH | 84 | |
| arg | 5 | 1 in 17 |
| his | 0 | 0 |
| lys | 5 | 1 in 17 |
| Glucagon | 180 | |
| arg | 16 | 1 in 11 |
| his | 4 | 1 in 45 |
| lys | 10 | 1 in 18 |
| Calcitonin | 93 | |
| arg | 6 | 1 in 16 |
| his | 3 | 1 in 31 |
| lys | 5 | 1 in 19 |
| ACTH | 41 | |
| arg | 3 | 1 in 14 |
| his | 1 | 1 in 41 |
| lys | 4 | 1 in 10 |
| Prolactin | 220 | |
| arg | 12 | 1 in 18 |
| his | 9 | 1 in 13 |
| lys | 11 | 1 in 11 |
| Interferon- | 133 | |
| alpha and beta | | |
| arg | 7 | 1 in 19 |
| his | 2 | 1 in 83 |
| lys | 7 | 1 in 19 |
| Interferon-gamma | 166 | |
| arg | 8 | 1 in 21 |
| his | 2 | 1 in 83 |
| lys | 21 | 1 in 8 |
| LH | 92 | |
| arg | 5 | 1 in 18 |
| his | 2 | 1 in 46 |
| lys | 7 | 1 in 13 |
| FSH | 129 | |
| arg | 5 | 1 in 26 |
| his | 2 | 1 in 65 |
| lys | 9 | 1 in 14 |
| CSF | 144 | |
| arg | 6 | 1 in 24 |
| his | 3 | 1 in 48 |
| lys | 6 | 1 in 24 |
| GH domain AOD9604 | 16 | |
| arg | 2 | 1 in 8 |

The preferred complexing agents are selected from the group consisting of arginine, lysine and tertiary substituted amines, such as those according to the following formula:

NR¹R²R³
wherein R¹ is chosen from the group comprising straight or branched chain mixed alkyl radicals from C6 to C22 and carbonyl derivatives thereof;
R² and R³ are chosen independently from the group comprising H, CH₂COOX, CH₂CHOHCH₂SO₃X, CH₂CHOHCH₂OPO₃X, CH₂CH₂COOX, CH₂COOX, CH₂CH₂CHOHCH₂SO₃X or CH₂CH₂CHOHCH₂OPO₃X and X is H, Na, K or alkanolamine provided R² and R³ are not both H; and
wherein when R¹ is RCO then R² may be CH₃ and R³ may be (CH₂CH₂)N(C₂H₄₀H)—H₂CHOPO₃ or R² and R³ together may be N(CH₂)₂N(C₂H₄₀H)CH₂COO—.

Preferred complexing agents include arginine, lysine or lauryliminodipropionic acid where complexation occurs between the alkaline nitrogen centre and the phosphoric acid ester to form a stable complex.

The phosphate derivative of the electron transfer agent may be administered to humans or animals through a variety of dose forms such as supplements, enteral feeds, parenteral dose forms, suppositories, oral dose forms, pulmonary and nasal delivery forms, dermal delivery including patches and creams.

For example, the phosphate derivative of the electron transfer agent may be administered by an orally or parenterally administered dose form. These include, tablets, powders, chewable tablets, capsules, oral suspensions, suspensions, emulsions or fluids, children's formulations, enteral feeds, nutraceuticals, and functional foods.

The dose form may further include any additives routinely used in preparation of that dose form such as starch or polymeric binders, sweeteners, coloring agents, emulsifiers, coatings and the like. Other suitable additives will be readily apparent to those skilled in the art.

In one embodiment, the dose form has an enteric coating as disclosed in international patent application PCT/AU01/01206, incorporated herein by reference.

In another embodiment, the dose form is a topical formulation as disclosed in international patent application PCT/AU02/01003, incorporated herein by reference.

The dose form may contain other pharmaceutical compounds which do not antagonise the activity of the phosphate derivatives of electron transfer agents. The other pharmaceutical compound may be administered before, with or after the one or more phosphate derivatives of one or more electron transfer agents. Preferably, the other pharmaceutical compounds are drugs for heart and cardiovascular disease and hypercholesterolaemic or dislipidaemic compounds. More preferably, the other pharmaceutical compounds are selected from the group consisting of cholesterol absorption inhibitors such as ezetimibe, cholesterol ester transfer protein inhibitors such as torcetrapib, other HDL increasing pharmaceutical compounds, statins, phosphate derivatives of statins and mixtures thereof. Examples of appropriate statins include provastatin, lovastatin and atorvastatin and phosphates thereof.

Preferably, the subject is an animal. More preferably, the animal is a mammal. Most preferably, the mammal is a human.

### DRAWINGS

Various embodiments/aspects of the invention will now be described with reference to the following drawings in which:

FIG. 1 is shows the results of Example 1 at 2 weeks.

FIG. 2 shows the results of Example 1 at 4 weeks.

FIG. 3 shows the results from Example 2.

### EXAMPLES

Various embodiments/aspects of the invention will now be described with reference to the following non-limiting examples.

### Example 1

This example evaluates the potential anti-CVD effects of a tocopheryl phosphate mixture in a well accepted CVD mouse model, the apolipoprotein E (APOE) mouse. The anti-CVD effects are assessed by decreases in the elevated plasma cholesterol, triglyceride and LDL levels.

The APOE knockout mouse model has been widely used in cardiovascular research as it mimics many of the properties observed clinically as part of the human disease. The APOE knockout mouse displays elevated circulating lipid levels from about 6 months of age. Placing these animals on a high fat, high cholesterol diet (i.e. 21% fat, 0.15% cholesterol) exacerbates the CVD, and therefore these symptoms are observed sooner.

### Methods

Animals: Male APOE knockout mice (15-20 g) were obtained from the Animal Resource Centre, Perth, Australia. They were fed a vitamin E stripped diet that contained 21% fat and 0.15% cholesterol rodent pellets from Glen Forrest Stockfeeders, W.A., Australia The mice were housed in standard laboratory cages with natural lighting, and acclimatised for at least 7 days before use.

In the current study the APOE mice were placed on a high fat, high cholesterol diet for a total of 8 weeks. Four weeks into this diet the animals are treated daily, via oral gavage, with either vehicle (1% CMC), tocopherol acetate (TA) at 100 mg/kg, or TP mixture (TPm) at 33.25, 66.5 or 133 mg/kg. The assessment of any improvement in the CDV of these mice involved blood being taken at regular intervals during the treatment, to assess the lipid levels and sectioning of the aortic arch, for assessment of the plaque formation at the end of the treatment period.

### Reagents:

Tocopherol Acetate (TA) (Sigma catalogue no. T-3001)

Tocopheryl phosphate mixture (TPm) containing monotocopheryl phosphate and ditocopheryl phosphate in a ratio of 2:1 (made in house batch no. SGNaTPm/21-10-04)

Carboxymethylcellulose (Sigma catalogue no. C-5678)

Milli Q water (in-house supply)

Formulation Preparation Solutions of TPm and TA were prepared in 1% carboxymethylcellulose (CMC) at the following concentrations: for TA, 15 mg/ml; and for TPm, 4.99, 9.98 and 19.95 mg/ml for dosing mice at TA 100 mg/kg and TPm at 33.25, 66.5 and 133 mg/kg, respectively. In preparing these solutions, the appropriate amount of each compound was made up in the 1% CMC and then placed in a water bath sonicator with warm water (about 50° C.) for 15 minutes. (The TPm dose of 133 mg/kg is used as the TA 100 mg/kg equivalent dose. Therefore the subsequent 66.5 and 33.25 mg/kg doses are 50 and 25% equivalent TA 100 mg/kg doses, respectively).

Dosing: Mice were weighed weekly, and the doses of each compound were calculated based on this weight for that dosing week. The animals were dosed between 7:30 am-11:00 am, each morning of the treatment period with a stainless steel gavage needle.

Blood collection: On 3 occasions the mice were restrained firmly and the tail nicked and about 50 μl of blood was collected into Capiject™ tubes. The tubes were then centrifuged at 8,000×g and the plasma collected, for lipid analysis (i.e. total cholesterol, triglyceride, HDL and LDL measurements, carried out by Gribbles Pathology).

The mice were bled prior to the commencement of the study (pre-bleeds), at the end of 4 weeks on the respective diets (prior to the commencement of the compound treatments) and 2 weeks into the treatment period. At the end of the treatment period, blood was collected from the animals directly from the heart after having been sacrificed by CO₂ asphyxiation. The APOE mice placed on the high fat and high cholesterol diet, increased 2-3 fold their plasma cholesterol, LDL and triglyceride levels during this feeding period This level was considered very high, as these mice already have quite elevated fasting lipid levels without being placed on an atherogenic diet. This was considered a good starting point in assessing the effectiveness of TPm to reduce these elevated lipid levels.

Analysis of plasma triglyceride: Measurement of plasma triglyceride took place with the use of the Triglyceride Determination Kit (Sigma, Catalogue No. TR0100). The procedure involves enzymatic hydrolysis by lipase of the triglycerides to glycerol and free fatty acids. The glycerol produced is then measured by coupled enzyme reactions shown below:

| | |
|---|---|
| Lipoprotein lipase | 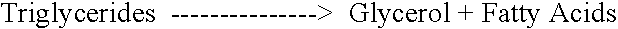 |
| Glycerol kinase | 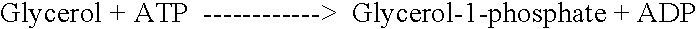 |
| Glycerol phosphate oxidase | 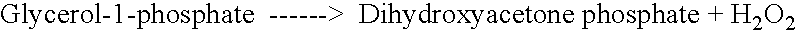 |
| Peroxidase | 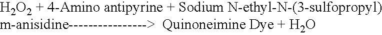 |

The reagents in the kit are prepared as per the manufacturers directions. The free glycerol standard reagent and samples are warmed to room temperature. A set of cuvets are prepared for Blank, Standard and Samples. 0.8 ml of Free Glycerol reagent is added to each cuvet, followed by 10 μl of water, glycerol standard or plasma, respectively. The samples are mixed, by inversion, and incubated at 37° C. for 5 minutes. The absorbance is then read at 540 nm and recorded as initial absorbance (IA). 0.2 ml of triglyceride reagent is then added to each cuvet and they are again mixed by inversion, and incubated for a further 5 minutes at 37° C. The final absorbance (FA) is then read and recorded at 540 nm. The total triglyceride concentration in plasma is then calculated are follows:

$$Total triglyceride = \frac{( \begin{matrix}{FA}_{sample} - \\ {FA}_{blank}\end{matrix} )}{( \begin{matrix}{FA}_{standard} - \\ {FA}_{blank}\end{matrix} )} ⋆ Concentration of standard$$

Analysis of plasma cholesterol: Measurement of plasma cholesterol took place with the use of the Infinity™ Cholesterol Reagent Kit (Thermo Electron Corp., Catalogue No. TR13521). The reagent is based on the following reactions:

| | |
|---|---|
| Cholesterol esterase | 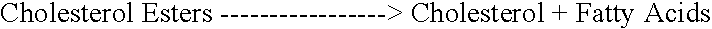 |
| Cholesterol oxidase | 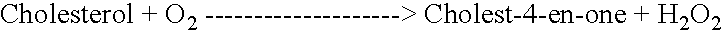 |
| Peroxidase | 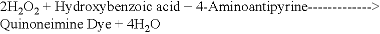 |

(1). Cholesterol esters are enzymatically hydrolysed by cholesterol esterase to cholesterol and free fatty acids.
(2). Free cholesterol, including that originally present, is then oxidized by cholesterol oxidase to cholest-4-en-3-one and hydrogen peroxide.
(3). The hydrogen peroxide combines with HBA and 4-aminoantipyrine to form a chromophore (quinoneimine dye) which may be quantitated at 500-550 nm.

The plasma is incubated with Cholesterol ReagentT™ (1:100). For example a sample volume of 3 μl is incubated with 300% of Cholesterol Reagent™, in a microtitre plate and incubated at 37° C. for 5 minutes. This is conducted for a calibrator also supplied in the kit.

The total cholesterol is then calculated as follows:

$$Cholesterol = Δ \frac{Δ Abs / min of unknown}{Abs / min of calibrator} ⋆ Calibrator concentration$$

### Example

Absorbance of calibrator=0.35
Absorbance of unknown=0.25
Value of calibrator=7.0 mmol/L
Cholesterol=0.25/0.35×7.0=5.0 mmol/L

Analysis of plasma HDL: Measurement of HDL took place with the use of the Infinity™ HDL Cholesterol Reagent Kit (Thermo Electron Corp., Catalogue No. TR39601).

The plasma (4 μl) is placed in a microtitre plate, and is incubated at 37° C. for 5 minutes with 300 μl of Reagent 1, followed by further 3 minute incubation after the addition of 100 μl of Reagent 2. The absorbance is then read at 600 nm. As for the cholesterol kit a calibrator also supplied in the kit is used also for the calculation.

Analysis of plasma LDL: Measurement of LDL took place with the use of the Infinity™ LDL Cholesterol Plus Reagent Kit (Thermo Electron Corp., Catalogue No. 3365-030).

The plasma (4 μl) is placed in a microtitre plate, and is incubated at 37° C. for 5 minutes with 300 μl of Reagent 1, followed by further 5 minute incubation after the addition of 100 μl of Reagent 2. The absorbance is then read at 600 nm. As for the cholesterol kit a calibrator also supplied in the kit is used also for the calculation.

Statistical Analysis Results are expressed as mean±SD. A Student's t-test was performed to determine whether there were significant differences in TA or TPm treated mice (whether it is cholesterol, triglyceride, HDL, LDL or plaque size) compared to no treatment or vehicle control groups. For a study of this type P<0.05, (*) was considered significant.

### Results and Discussion

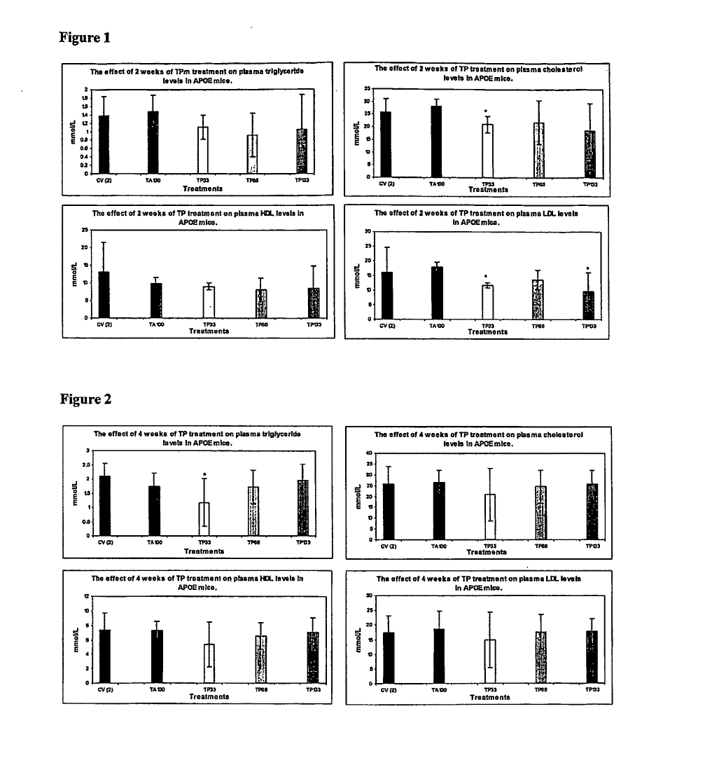

FIG. 1 shows the results obtained after 2 weeks of treatment.

FIG. 2 shows the results obtained after 4 weeks of treatment.

The administration of TPm, in particular at 33.25 mg/kg, gave a significant decrease in plasma total cholesterol and LDL concentrations compared to no treatment or vehicle alone treated mice, after 2 weeks of treatment. Following the 4 weeks of treatment, the 33.25 mg/kg dose of TPm still provided a significant decrease in plasma triglyceride levels compared to no treatment or vehicle alone controls. These results suggest that TPm (in particular at 33.25 mg/kg) is potentially effective in lowering elevated cholesterol, triglyceride and LDL levels circulating in blood.

### Example 2

This example evaluated the effects of a tocopheryl phosphate mixture (TPm) (mono-tocopheryl phosphate and di-tocopheryl phosphate) on the development of atherosclerotic lesions in male APOE deficient mice.

### Methodology

Twenty-eight mice were divided into 4 groups: 2 control groups, a tocopherol acetate (TA) group (150 mg TA/kg feed) and a TPm group (200 mg TPm/kg feed containing 7% fat).

### Diets:

‘Induction phase’—The induction phase consisted of the first 16 weeks of the treatment period. During this period the animals were fed a mouse pellet diet low in vitamin E (containing less than 20 mg vitamin E per kg food, with a 7% total fat; modified version of the standard AIN93G rodent diet (SF05-040, Specialty feeds, Glen Forrest, Wash. Australia). Control animals were fed the diet alone, while TA-feed contained 150 mg TA/kg feed and TPm-feed contained 200 mg TPm/kg feed. These feeds delivered on average doses of 21 and 26 mg/kg body weight, respectively. The 26 mg/kg TPm dose was calculated to be the tocopherol equivalence of the TA dose.

‘Challenge phase’—This phase consisted of the final 8 weeks of the treatment period. During this period the animals were fed a low vitamin E, high fat (21%), high cholesterol (0.15%) specialised rodent pellet diet (HFHC; SF04-055 mouse diet is a version of the standard SF00-219 diet containing less than 20 mg vitamin E per kg; Specialty feeds, Glen Forrest, Wash., Australia).

The 4 groups of animals were placed on the diet regimes outlined in the table below:

| Treatment Group | Diet for weeks 0-16 | Diet for weeks 16-24 |
|---|---|---|
| Control (C24) | SF05-040 | SF05-040 |
| Control (C16/8) | SF05-040 | SF04-055 |
| TA | SF05-040 + TA (150 mg | SF04-055 + TA |
| | TA/kg feed) | (150 mg TA/kg feed) |
| TPm | SF05-040 + TPm | SF04-055 + TPm |
| | (200 mg TPm/kg feed) | (200 mg TPm/kg) |

Of the two control animal groups only one was placed on the SF04-055, HFHC diet, while the other control group was maintained on the SF05-040 mouse pellet diet (only 7% fat) for the entire treatment period 24 week treatment period (C24). This was done so as to establish the effect of the HFHC diet alone on the various atherosclerotic parameters being measured, and to assess whether or not treatments with the various compounds were as good as animals maintained on normal diets. TA treated mice were fed pellets with 150 mg TA/kg feed and TPm treated mice were fed the pellets with 200 mg TPm/kg feed. These feeds delivered doses averaging 21 and 26 mg/kg body weight, respectively. The 26 mg/kg TPm dose was calculated to be the tocopherol equivalent to the TA dose.

During the induction phase, the control mice developed mild hypercholesterolemia and atherosclerotic lesions. After 16 weeks of treatment with TPm, the mice showed a 34% reduction in total cholesterol (1.44+/−1.37 vs 17.38+/−1.47 mmol/L), 51% reduction in triglycerides (0.99+/−0.14 vs 2.00+/−0.58 mmol/L) and a 44% reduction in LDL-C (4.67+/−0.70 vs 8.38+/−0.76 mmol/L) compared to control animals. These reductions were significantly different from control animals, and were far greater than those seen with TA treatment.

After the challenge phase, the control mice developed severe hypercholesterolemia and advanced atherosclerotic lesions. The TA-treated mice showed no significant reduction in plasma lipid levels or evidence for lesion regression; although there was an average 12% decrease in lesion area (this was not significant). However, the TPm treatment gave a reduction of 15% in total cholesterol (43.8+/−4.38 vs 37.08+/−5.15 mmol/L), 28% reduction in triglycerides (1.63+/−0.22 vs 2.27+/−0.20 mmol/L) and 16% reduction in LDL-C (15.02+/−2.61 vs 17.95+/−1.51 mmol/L) as well as a significant reduction (58%) in aortic lesion formation.

TABLE 1
Mouse total cholesterol level comparisons (mean ± SD; mmol/L)
during the induction and challenge phases of the study.

| | Baseline | Induction phase | Challenge phase |
|---|---|---|---|
| | Week | | |
| Group (n) | 0 | 16 | 24 |
| Control SF05-040 diet | 11.96 ± 0.82 | 13.43 ± 2.21 | 11.43 ± 1.89 |
| alone (n = 8-12) | (n = 12) | (n = 12) | (n = 8) |
| Control (n = 8) | 12.80 ± 1.35 | 17.38 ± 1.47 | 43.8 ± 4.38# |
| TA (n = 8) | 12.75 ± 1.41 | 14.47 ± 1.32 | 47.13 ± 4.44 |
| TP (n = 8) | 11.61 ± 1.24 | 11.44 ± 1.37* | 37.08 ± 5.15* |

#indicates control mice maintained on HFHC diet (for the final 8 weeks of the treatment) had significantly higher plasma cholesterol levels to control mice on the normal diet (P < 0.05).
*indicates significance (P < 0.05) from Control animals.

TABLE 2
Mouse triglyceride level comparisons (mean ± SD; mmol/L) during
the induction and challenge phases of the study.

| | Baseline | Induction Phase | Challenge Phase |
|---|---|---|---|
| | Week | | |
| Group (n) | 0 | 16 | 24 |
| Control SF05-040 | 1.73 ± 0.13 | 1.10 ± 0.25 | 1.45 ± 0.21 |
| diet | | | |
| alone (n = 8-12) | (n = 12) | (n = 12) | (n = 8) |
| Control (n = 8) | 1.18 ± 0.28 | 2.00 ± 0.58 | 2.27 ± 0.20# |
| TA (n = 8) | 1.75 ± 0.25 | 1.30 ± 0.22* | 2.23 ± 0.29 |
| TP (n = 8) | 1.54 ± 0.38 | 0.99 ± 0.14* | 1.63 ± 0.22* |

#indicates control mice maintained on HFHC diet (for the final 8 weeks of the treatment) bad significantly higher plasma cholesterol levels to control mice on the normal diet (P < 0.05).
*indicates significance (P < 0.05) from Control animals.

TABLE 3
Mouse LDL-C level comparisons (mean ± SD; mmol/L) during the
induction and challenge phases of the study.

| | Baseline | Induction Phase | Challenge Phase |
|---|---|---|---|
| | Week | | |
| Group (n) | 0 | 16 | 24 |
| Control SF05-040 | 6.83 ± 0.54 | 7.65 ± 0.54 | 5.20 ± 0.96 |
| diet | | | |
| alone (n = 8-12) | (n = 12) | (n = 12) | (n = 8) |
| Control (n = 8) | 8.08 ± 1.20 | 8.38 ± 0.76 | 17.95 ± 1.51# |
| TA (n = 8) | 8.85 ± 1.66 | 6.51 ± 0.71 | 18.98 ± 2.29 |
| TP (n = 8) | 7.84 ± 1.51 | 4.67 ± 0.70* | 15.02 ± 2.61 |

#indicates control mice maintained on HFHC diet (for the final 8 weeks of the treatment) had significantly higher plasma cholesterol levels to control mice on the normal diet (P < 0.05).
*indicates significance (P < 0.05) from Control animals.

TABLE 4
Atheromatous lesion area (mean ± SD, % lesion coverage), at the
end of the treatment period.

| Group (n) | % Lesion coverage |
|---|---|
| Control SF05-040 diet alone (n = 8) | 8.9 ± 1.7 |
| Control C16/8 (n = 8) | 10.7 ± 1.3 |
| TA (n = 8) | 9.4 ± 1.1 |
| TPm (n = 8) | 4.5 ± 1.3* |

*indicates significance (P < 0.05) from Control animals.

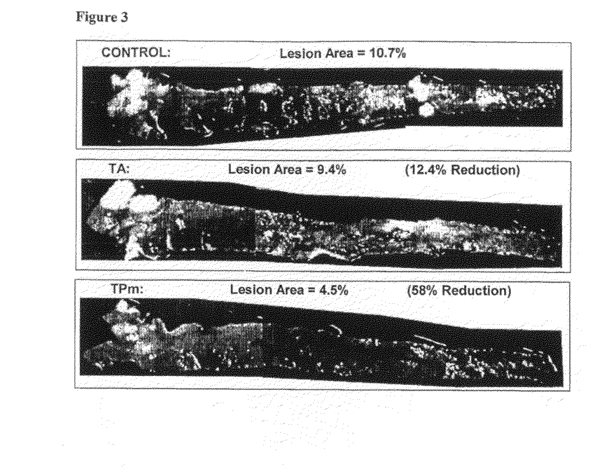

FIG. 3 shows the aortic lesion formation assessment of aortae by Oil red O staining.

The aortic root, thoracic and abdominal aortae were stained with Oil red O (ORO), (which stains lipids red), showed substantial lipid deposits in vascular atherosclerotic lesions. Table 4 shows the lesion sizes at the end of the 24 week treatment period across each of the treatment groups. On average at the end of the induction period (at week 16) each mouse had approximately 5% atheromatous lesions coverage per aortic region, (data not shown). The lesion area was increased to 8.9% by the end of the 24 week period in animals maintained on the induction diet alone (7% fat alone). Animals that were placed on the atherogenic diet for the final 8 weeks showed on average of 10.7% atheromatous lesions per aortic region compared to 9.4% in TA and 4.5% in TPm treated mice. TA treatment saw a 12% improvement in atherosclerotic lesions (this was not statistically significant), while TPm treatment saw a significant 58% reduction in lesion formation compared to control mice maintained on the same diet regime.

### CONCLUSION

The findings show a significant reduction in the lipid profiles (LDL, total cholesterol and triglyceride) in animals treated with TPm, indicating that TPm treatment may treat hyperdyslipidemia and related diseases. As a secondary outcome, the findings show a significant decrease in the atherosclerotic lesion size in TPm treated APO E-deficient mice, indicating that TPm treatment may treat or slow the progression of atherosclerotic lesions in this mouse strain.

The word ‘comprising’ and forms of the word ‘comprising’ as used in this description and in the claims does not limit the invention claimed to exclude any variants or additions.

Modifications and improvements to the invention will be readily apparent to those skilled in the art. Such modifications and improvements are intended to be within the scope of this invention.

The claims defining the invention are as follows:

1. A therapy for lowering the blood levels of a lipid selected from the group comprising LDL cholesterol, triglycerides, overall cholesterol and mixtures thereof, the therapy comprising the step of administering an effective amount of one or more phosphate derivatives of one or more electron transfer agents; wherein the phosphate derivative of an electron transfer agent is not ascorbyl-tocopheryl phosphate.

2. The therapy according to claim 1 wherein the electron transfer agent is selected from the group consisting of tocols, retinol, vitamin K1 and mixtures thereof.

3. The therapy according to claim 2 wherein the electron transfer agent is selected from the group consisting of tocols and mixtures thereof.

4. The therapy according to claim 3 wherein the electron transfer agent is selected from the group consisting of α-tocopherol, δ-tocopherol, γ-tocopherol, α-tocotrienol, δ-tocotrienol, γ-tocotrienol and mixtures thereof.

5. The therapy according to claim 4 wherein the phosphate derivatives of electron transfer agents is selected from the group consisting of mono-tocopheryl phosphate, di-tocopheryl phosphate, mono-tocotrienyl phosphate, di-tocotrienyl phosphate and mixtures thereof.

6. The therapy according to claim 5 wherein the phosphate derivatives of electron transfer agents is a mixture of mono-tocopheryl phosphate and di-tocopheryl phosphate.

7. The therapy according to claim 6 further comprising the step of administering one or more other pharmaceutical compounds which do not antagonise the activity of the phosphate derivative of an electron transfer agent.

8. The therapy according to claim 7 wherein the other pharmaceutical compounds are selected from the group consisting of cholesterol absorption inhibitors, cholesterol ester transfer protein inhibitors, HDL increasing pharmaceutical compounds, statins, their phosphate derivatives and mixtures thereof.

9. A therapy for lowering the blood levels of a lipid selected from the group consisting of LDL cholesterol, triglycerides, overall cholesterol and mixtures thereof, the therapy comprising the step of administering an effective amount of one or more prodrugs of one or more phosphate derivatives of one or more electron transfer agents; wherein the phosphate derivative of an electron transfer agent is not ascorbyl-tocopheryl phosphate.

10. A therapy of alleviating symptoms, treating or preventing a disease selected from the group consisting of cardiovascular disease, atherosclerosis, diabetes mellitus, chronic renal disease, primary and secondary hyperlipidemias and dyslipidemia, retinopathies, liver and spleen enlargement, xanthomas and combinations thereof, the therapy comprising administering a pharmaceutical formulation comprising an effective amount of one or more phosphate derivatives of one or more electron transfer agents to a subject having or at risk of developing the disease; wherein the phosphate derivative of an electron transfer agent is not ascorbyl-tocopheryl phosphate.

11. Use of an effective amount of one or more phosphate derivatives of one or more electron transfer agents together with a suitable carrier or diluent in the manufacture of a medicament for lowering the blood levels of a lipid selected from the group consisting of LDL cholesterol, triglycerides, overall cholesterol and mixtures thereof; wherein the phosphate derivative of an electron transfer agent is not ascorbyl-tocopheryl phosphate.

12. A pharmaceutical composition when used for lowering the blood levels of a lipid selected from the group consisting of LDL cholesterol, triglycerides, overall cholesterol and mixtures thereof, the composition comprising an effective amount of one or more phosphate derivatives of one or more electron transfer agents; wherein the phosphate derivative of an electron transfer agent is not ascorbyl-tocopheryl phosphate.
